(19) United States
(12) Patent Application Publication
(10) Pub. No.: US 2006/0089535 A1
(43) Pub. Date: Apr. 27, 2006

(54) Piston-actuated endoscopic steering system

(76) Inventors: Dan Raz, Haifa (IL); Reuven Nir, Merhavia (IL); Yakov Baror, Vardia (IL)

Correspondence Address: BRUCE E. LILLING;LILLING & LILLING P.C., P.O. BOX 560, GOLDEN BRIDGE, NY, 10526, US

(21) Appl. No.: 10/520,828

(22) PCT Filed: Jul. 8, 2003
(86) PCT No.: PCT/IL03/00564

§ 371 Date: Sep. 12, 2005

Related U.S. Application Data
(60) Provisional application No. 60/395,694, filed on Jul. 11, 2002.

(51) Int. Cl. A61B 1/00

(52) U.S. Cl. 600/152; 600/146

(57) ABSTRACT

Endoscopic steering apparatus is provided, including an endoscope (80) having distal (82) and proximal (98) ends thereof. In a preferred embodiment, at least one proximal cylinder (92) is disposed in a vicinity of the proximal end of the endoscope, and at least one proximal piston (94) is slidably coupled to the at least one proximal cylinder. This piston is typically manually driven. A first distal cylinder (88) is disposed at the distal end of the endoscope, and a first distal piston (90) is slidably coupled to the first distal cylinder. A second distal cylinder (88) is disposed at the distal end of the endoscope, and a second distal piston (90) is slidably coupled to the second distal cylinder. A first tube (86) is coupled to the first distal cylinder and to the at least one proximal cylinder, and a second tube (86) is coupled to the second distal cylinder and to the at least one proximal cylinder. A linkage (96) is disposed at the distal end of the endoscope and coupled to the first distal piston and to the second distal piston, such that displacement of at least one of the distal pistons causes displacement of the linkage and steering of the distal end of the endoscope.

### CROSS-REFERENCES TO RELATED APPLICATIONS

This application claims priority from U.S. Provisional Patent Application 60/395,694 to Raz et al., filed Jul. 11, 2002, entitled, “Piston-actuated endoscopic steering system,” which is assigned to the assignee of the present patent application and is incorporated herein by reference.

### FIELD OF THE INVENTION

The present invention relates generally to the steering of flexible medical devices, and specifically to methods and devices for steering endoscopes during medical procedures.

### BACKGROUND OF THE INVENTION

The use of an endoscope for examining a body cavity is well known in the art. The diagnostic and therapeutic advantages conferred by direct examination of the gastrointestinal tract with a flexible endoscope have made this method a standard procedure of modern medicine. One of the most common endoscopic procedures is colonoscopy, which is performed for a wide variety of purposes, including diagnosis of cancer, determination of the source of gastrointestinal bleeding, viewing a site affected by inflammatory bowel disease, removing polyps, and reducing volvulus and intussusception.

While colonoscopy is useful and effective, it is a difficult procedure for a physician to perform and is painful and occasionally dangerous for the patient. These problems stem from the need to push and steer the long, flexible colonoscope through the intestine by pushing it in from its proximal end, outside the body.

The gastrointestinal tract follows a tortuous path with many sharp turns, sometimes making it difficult or impossible to advance an endoscope to a desired site. A complication of colonoscopy arises when the colonoscope perforates the colon, typically at a sharp turn, leading to spillage of bowel contents into the abdominal cavity, which may lead to infection in the abdominal cavity and the need for emergency surgery. The ability to easily steer an endoscope around sharp turns in the gastrointestinal tract would extend the region of the gastrointestinal tract that is amenable to visualization and/or treatment by the endoscope, greatly enhancing diagnosis and treatment of gastrointestinal diseases. A number of methods and devices have been proposed for this purpose, but the region of the gastrointestinal tract that can be accessed via endoscopes is still limited by the difficulty of navigating around sharp bends.

A common means for steering flexible endoscopes is to connect guided cables or wires to the distal end of the endoscope. Tension is applied by a physician at the proximal end to the cables or wires, in order to induce a desired bend at the distal end. The extent to which the endoscope can be steered by this technique is limited by friction between each wire and a sheath surrounding the wire. In particular, if a physician only needs to overcome a single turn in the gastrointestinal tract, then the force F1 that must be applied at the proximal end in order to generate a force F2 at the distal end can be approximated as F1=F2*e^{μα}, where μ is the coefficient of friction between the wire and the sheath, and α is the effective angle defined by the turn in the gastrointestinal tract. If, as is common, the endoscope travels through a number i of turns αᵢ in the gastrointestinal tract, then the total force can increase significantly (and often prohibitively) to
F1=F2*e^{μΣ|α}^{ᵢ}^{|}.

To overcome the effects of friction incurred using wire-based steering systems, attempts have been made to introduce hydraulic steering to endoscopes, but none of these have been commercially viable, because all prior art hydraulic steering systems known to the inventor are complicated, expensive, bulky and/or require external power or pressure sources, as well as the equipment to manage these sources. Because of these drawbacks, only wire-based techniques are currently used for endoscopic steering applications.

U.S. Pat. No. 3,773,034 to Burns et al., which is incorporated herein by reference, describes a method for steering a catheter through a body passage by selectively pressurizing fluid conduits that run along the outside of the catheter. Pressurizing a conduit on one side of the catheter results in a slight elongation of that side of the catheter, due to elastic deformation, while the length of the opposing side stays constant, resulting in a curvature of the catheter.

U.S. Pat. No. 4,483,326 to Yamaka et al., which is incorporated herein by reference, describes a method for steering a flexible endoscope by selectively applying tension to control wires connected to the distal end of the endoscope. Tension is applied to the wires by rotating a drum, about which the wires are wound.

U.S. Pat. No. 4,559,928 to Takayama, which is incorporated herein by reference, describes a method for steering a flexible endoscope by selectively applying tension to control wires connected to the distal end of the endoscope via a motor.

U.S. Pat. No. 4,721,099 to Chikama, U.S. Pat. No. 5,957,863 to Koblish et al., and U.S. Pat. No. 5,297,443 to Wentz, which are incorporated herein by reference, describe a method for steering a flexible endoscope by selectively applying tension to control wires connected to the distal end of the endoscope via a hydraulic actuator at the proximal end.

U.S. Pat. No. 4,700,693 to Lia et al. and U.S. Pat. No. 4,790,294 to Allred III et al., which are incorporated herein by reference, describe a method for steering a flexible endoscope by selectively applying tension to control cables connected to the distal end of the endoscope. The control cables run through peripheral bores in axially-aligned flat washers and spacer beads. The degree of bending can be controlled via the spacing of the washers and beads, as the beads act as hinges.

U.S. Pat. No. 6,051,008 to Saadat et al. and U.S. Pat. No. 5,188,111 to Yates et al., which are incorporated herein by reference, describe a method for steering a flexible endoscope by selectively heating or cooling strips composed of shape-memory alloy near the distal end of the endoscope.

U.S. Pat. Nos. 4,991,957, 5,048,956 and 5,096,292 to Sakamoto et al., which are incorporated herein by reference, describe methods for steering a flexible endoscope by selectively ejecting pressurized fluid from one or more jet ports near the distal end of the apparatus.

U.S. Pat. No. 5,577,992 to Chiba et al., U.S. Pat. No. 5,018,506 and U.S. Pat. No. 5,203,319 to Danna et al., U.S. Pat. No. 4,794,912 to Lia, U.S. Pat. No. 5,140,975 to Krauter, and U.S. Pat. No. 4,983,165 to Loiterman, which are incorporated herein by reference, describe methods for steering a flexible endoscope by selectively pressurizing balloons or bladders near the distal end of the endoscope to either push the end of the endoscope in a preferred direction or distort the tip of the endoscope to provide a desired curvature.

U.S. Pat. No. 4,890,602 to Hake, which is incorporated herein by reference, describes methods for steering a flexible endoscope by selectively inflating longitudinal tubes along the endoscope to control the curvature of the endoscope. The rigidity of the endoscope is also controlled by the degree of inflation of the longitudinal tubes.

U.S. Pat. No. 5,314,428 to Marotta, which is incorporated herein by reference, describes a method for steering a flexible catheter by selectively pressurizing one or more of multiple channels at the tip of the catheter with fluid. Each channel is connected to a piston at the proximal end, which is activated by a rotary mechanical assembly.

U.S. Pat. No. 4,962,751 to Krauter, which is incorporated herein by reference, describes methods for steering endoscopes by using fluid dynamic “muscles,” bladders that contract longitudinally while expanding radially when pressurized, to apply forces to steering wires or cables. The fluid dynamic muscles are placed near the distal end of the endoscope, so that the steering wires or cables can be relatively short. The fluid dynamic muscles are connected to a source of pressurized fluid at the proximal end by a flexible tube.

U.S. Pat. No. 4,832,473 to Ueda and U.S. Pat. No. 5,179,934 to Nagayoshi et al, which are incorporated herein by reference, describe methods for steering endoscopes by using fluid dynamic muscles in a portion of the endoscope between the distal and proximal ends.

European Patent Application EP 1 036 539 A1 by Matasova, which is incorporated herein by reference, describes a method for steering a flexible catheter by including traction lines connecting the distal and proximal ends of the endoscope. A piston/cylinder connected to vacuum and pressure sources is used to supplement the forces delivered by the traction lines and aid in steering the endoscope.

A paper entitled, “A Micro Robotic Arm For A Self Propelling Colonoscope,” published in Proc. Actuator 98, 6th Int. Conf. on New Actuators, pp. 576-579, June 1998, which is incorporated herein by reference, describes a self-propelling endoscopic system for colonoscopy that comprises a flexible arm, which is controlled by shape memory alloy materials, to which are attached endoscopic tools. The endoscopic tools are controlled by either heating/cooling of shape memory alloy mechanisms, or by hydraulic means via a piston/cylinder apparatus. A simple piston/cylinder apparatus is used with a single pressure port on the cylinder, such that both positive and negative pressures must be used to operate an attached tool. Since only one atmosphere of negative pressure can be applied, forces applied by any tools are limited. Embodiments of the present invention specifically address and overcome this limitation.

### SUMMARY OF THE INVENTION

It is an object of some aspects of the present invention to provide an improved system and method for steering an object within a lumen.

It is a further object of some aspects of the present invention to provide an improved steering mechanism for steering an endoscope within a body cavity of a patient for purposes of examination, diagnosis, and treatment.

In preferred embodiments of the present invention, a distal section of a flexible endoscope is advanced through the gastrointestinal tract with the aid of a steering mechanism near the distal end of the endoscope. The steering mechanism comprises one or more cylinders, each containing a piston, wherein movement of one or more of the pistons actuates rods, wires and/or cables in the steering mechanism to cause turning of the distal end of the endoscope. Movement of the one or more pistons is achieved by introducing or removing fluid into/from the corresponding cylinders, so as to cause a motion of the piston. The fluid is delivered from the proximal end of the endoscope to the cylinders of the steering mechanism near the distal end of the endoscope via a closed system of flexible tubes.

Flexible tubes are used such that means for delivering fluid to and from the steering mechanism do not impede the advancement of the distal end of the endoscope past curves in the gastrointestinal tract. These embodiments of the present invention obviate the need for wires running the length of the endoscope to steer the endoscope, thus minimizing some of the difficulties involved with having wires along the entire endoscope, such as friction between the wires and the sleeve, and difficulty navigating sharp turns in the gastrointestinal tract.

Means for providing fluid to the cylinders in the steering mechanism via the flexible tubes are preferably located near the proximal end of the endoscope, external to the patient. Thus, there are not necessarily the same size restrictions as on the steering mechanism, which is near the distal end of the endoscope and is introduced into the patient's gastrointestinal tract via the endoscope.

In a preferred embodiment of the present invention, a drive-piston/cylinder system is used to provide pressure to the fluid in the flexible tubes, so as to drive the steering mechanism. Preferably, the operator uses hand and/or foot movements to displace one or more drive-pistons in their respective cylinders, resulting in movement of fluid into or out of the steering mechanism cylinders, and thus movement of the corresponding pistons and the desired steering of the distal end of the endoscope. Thus, physical forces applied by the operator are directly or proportionately applied to steer the endoscope, providing the operator with a sense of feedback. After a relatively small amount of training and practice, the operator typically learns the amount of force necessary to apply to a mechanical user-interface device such as a joystick, in order to turn the distal end of the endoscope a specified amount. The physical force required to steer the endoscope is controlled by leveraging or other aspects of the mechanical and/or hydraulic design of the drive mechanism.

In a preferred embodiment of the present invention, each steering mechanism cylinder comprises one port for introduction or withdrawal of fluid so as to move the corresponding piston. A piston divides each steering mechanism cylinder into two regions: (a) a fluid-transfer region, comprising a port through which fluid is actively added or withdrawn, and (b) a passive region, which may be open at one end, or which may comprise a spring, or a fixed amount of a compressible fluid. Preferably, the steering mechanism cylinder is aligned with the longitudinal axis of the endoscope, and the fluid-transfer region is closer to the distal end of the endoscope than the other region. This arrangement is preferred for some applications, because when fluid is added to the distal end of one of the steering mechanism cylinders, a tensile force will develop in wires of the steering mechanism that connect the piston to the steerable distal end of the endoscope, eliminating the possibility of buckling due to compressive loads. Mechanical linkages between two or more of the steering mechanism cylinders are preferably designed so as to maintain tensile loads in these steering mechanism wires when fluid is added to the fluid-transfer region of the cylinder. Alternatively or additionally, one or more suitably-configured rods are coupled to the steering mechanism cylinders so as to be placed in compression during application or removal of fluid in the fluid-transfer region of the cylinder(s), and to thereby facilitate steering of the endoscope.

For applications in which the passive region of each steering mechanism cylinder contains a compressible fluid (e.g., air), the fluid typically functions essentially as a spring, and acts to return the piston to its equilibrium position. Alternatively or additionally, this region comprises a solid spring to assist in returning the piston to its equilibrium position once no external pressure is applied to the cylinder.

In another preferred embodiment of the present invention, each steering mechanism cylinder comprises two ports, one on each side of the piston, which are coupled respectively to two fluid-transfer regions of the cylinder, into or out of which fluid is actively added or removed. Flexible tubes convey hydraulic pressure from the proximal end of the endoscope to each port. Movement of a given piston is initiated responsive to the difference in the fluid pressure on opposing sides of the piston. By regulating the pressure on each side of the piston, accurate control of the force delivered by the piston to the steering mechanism linkage is achieved. Inclusion of input/output ports in each region of the steering mechanism cylinders allows for the use of substantially incompressible fluids, e.g., water, to drive the steering mechanism.

There is therefore provided, in accordance with a preferred embodiment of the present invention, endoscopic steering apparatus, including:

an endoscope, having distal and proximal ends thereof;

at least one proximal cylinder, disposed in a vicinity of the proximal end of the endoscope;

at least one proximal piston, slidably coupled to the at least one proximal cylinder;

a first distal cylinder, disposed at the distal end of the endoscope;

a first distal piston, slidably coupled to the first distal cylinder;

a second distal cylinder, disposed at the distal end of the endoscope;

a second distal piston, slidably coupled to the second distal cylinder;

a first tube, coupled to the first distal cylinder and to the at least one proximal cylinder;

a second tube, coupled to the second distal cylinder and to the at least one proximal cylinder; and

a linkage disposed at the distal end of the endoscope and coupled to the first distal piston and to the second distal piston, such that displacement of at least one of the distal pistons causes displacement of the linkage and steering of the distal end of the endoscope.

The proximal piston is typically manually driven, but may also be driven by an actuator, e.g., an electromechanical actuator.

In a preferred embodiment, the apparatus is configured such that:
the at least one proximal cylinder includes respective first and second proximal cylinders,
the at least one proximal piston includes respective first and second proximal pistons, slidably coupled to the first and second proximal cylinders, respectively,
the first tube is coupled to the first distal cylinder and to the first proximal cylinder, and
the second tube is coupled to the second distal cylinder and to the second proximal cylinder.

Alternatively or additionally, the apparatus is configured such that:
the at least one proximal cylinder includes a single proximal cylinder,
the at least one proximal piston includes a single proximal piston, slidably coupled to the single proximal cylinder,
the single proximal cylinder has a proximal port and a distal port, disposed at respective ends of the single proximal cylinder, and
the first and second tubes are coupled to the single proximal piston at the proximal and distal ports, respectively.

For some applications, the linkage includes a flexible element, disposed in the endoscope such that tension in the element translates a displacement of one of the distal pistons into a change in angular disposition of the distal end of the endoscope. Alternatively or additionally, the linkage includes an element, disposed in the endoscope such that compression in the element translates a displacement of one of the distal pistons into a change in angular disposition of the distal end of the endoscope.

Preferably, the apparatus includes a mechanical user-interface device, which is coupled to the at least one proximal cylinder so as to mechanically transduce a force generated by a user of the steering apparatus into a motion of the at least one proximal piston.

There is also provided, in accordance with a preferred embodiment of the present invention, endoscopic steering apparatus, including:

an endoscope, having distal and proximal ends thereof, the distal end including a forward section and a rear section flexibly coupled to the forward section;

a distal cylinder, disposed at the rear section of the distal end of the endoscope;

a distal piston, slidably coupled to the distal cylinder and coupled to the forward section of the distal end of the endoscope;

a proximal cylinder, disposed in a vicinity of the proximal end of the endoscope;

a manually-driven proximal piston, slidably coupled to the proximal cylinder; and

a tube, coupled between the distal cylinder and the proximal cylinder, such that displacement of the proximal piston generates a pressure in the tube capable of displacing the distal piston and rotating the forward section with respect to the rear section.

Preferably, the distal cylinder has a distal port, distal to the distal piston, in communication with the tube, such that positive pressure in the tube responsive to displacement of the proximal piston induces proximal motion of the distal piston.

Further preferably, the apparatus is configured such that:
the proximal cylinder has a first port, which is in communication with a first face of the proximal piston, and a second port, which is in communication with a second face of the proximal piston,
the tube is coupled to the proximal cylinder at the first port, so as to be in communication with the first face of the proximal piston,
the distal cylinder has a proximal port, proximal to the distal piston, and
the apparatus includes an additional tube having distal and proximal ends thereof, the additional tube being in communication at the distal end thereof with the proximal port of the distal cylinder, and being in communication at the proximal end thereof with the second port of the proximal cylinder,
whereby positive pressure in the additional tube responsive to displacement of the proximal piston induces distal motion of the distal piston.

For some applications, the apparatus is configured such that:
the apparatus includes an additional proximal cylinder, disposed in a vicinity of the proximal end of the endoscope,
the apparatus includes an additional manually-driven proximal piston, slidably coupled to the additional proximal cylinder,
the distal cylinder has a proximal port, proximal to the distal piston, and
the apparatus includes an additional tube, coupled between the proximal port of the distal cylinder and the additional proximal cylinder,
whereby positive pressure in the additional tube responsive to displacement of the additional proximal piston induces distal motion of the distal piston.

The present invention will be more fully understood from the following detailed description of the preferred embodiments thereof, taken together with the drawings, in which:

### BRIEF DESCRIPTION OF THE DRAWINGS

FIGS. 1A and 1B are schematic, sectional drawings of an endoscope comprising a hydraulic steering mechanism, in two respective states thereof, according to a preferred embodiment of the present invention;

FIG. 2 is a schematic, sectional drawing of an endoscope comprising a hydraulic steering mechanism, according to another preferred embodiment of the present invention;

FIG. 3 is a schematic, sectional drawing of an endoscope comprising a hydraulic steering mechanism, according to yet another preferred embodiment of the present invention; and

FIG. 4 is a schematic, sectional drawing of an endoscope comprising a hydraulic steering mechanism, according to still another preferred embodiment of the present invention.

### DETAILED DESCRIPTION OF PREFERRED EMBODIMENTS

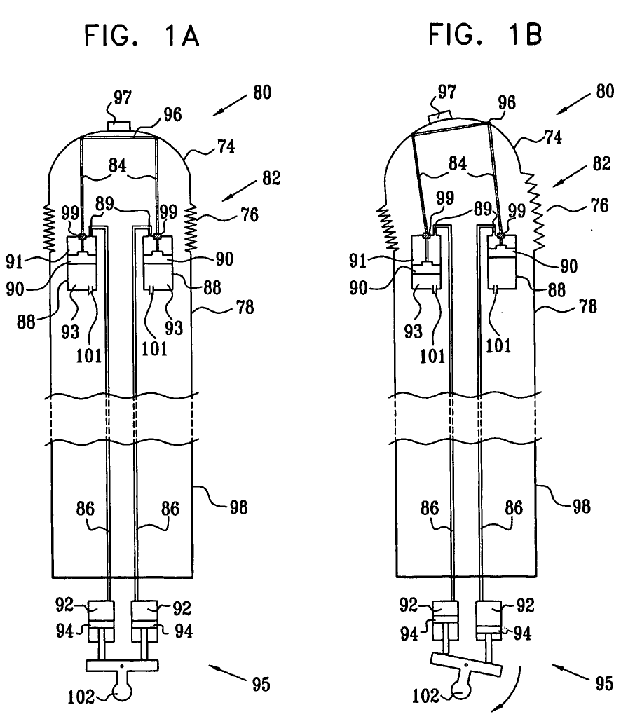

Reference is now made to FIGS. 1A and 1B, which are schematic sectional drawings of a flexible endoscope 80 comprising a hydraulic steering mechanism, in accordance with a preferred embodiment of the present invention. The endoscope is shown in a first, straight, state (FIG. 1A), as well as in a second state (FIG. 1B) during steering of the endoscope. Endoscope 80 comprises a distal portion 82, which is advanced into the gastrointestinal tract of a patient, and a proximal portion 98, part of which remains external to the patient and is accessible to the operator of the endoscope. Typically, a probe 97 is disposed near the tip of endocope 80, for diagnosis and/or treatment of the patient. In a preferred embodiment, probe 97 comprises a camera for examining the interior of the gastrointestinal tract. Alternatively or additionally, probe 97 comprises a tool, such as a biopsy device or a treatment device.

Distal portion 82 preferably comprises a forward section 74 coupled to articulate at a joint 76 with a rear section 78. Means for facilitating steering of the endoscope are located in rear section 78, and preferably comprise a plurality of cylinders 88, each of which comprises a piston 90. It is to be understood that a universal joint or a variety of other joint structures known in the art may be used for coupling forward section 74 to rear section 78, and that the scope of the present invention is not limited to a joint such as that shown in the figures. For clarity, FIGS. 1A and 1B show only two cylinders 88, disposed at 180 degrees with respect to the central axis of the endoscope, in order to allow for turning of the endoscope in an arc. However, it is to be understood that the scope of the present invention includes the use of devices with more than two cylinders disposed at respective positions around the central axis, so as to allow the operator to turn the endoscope in any direction. Alternatively, two cylinders may be placed on the same side of the central axis, e.g., at 12 o'clock and 3 o'clock, and controlled simultaneously to produce x-axis and y-axis motion. Further alternatively, only one cylinder may be located in rear section 78, and coupled to facilitate the steering of forward section 74.

Each piston 90 is preferably coupled to one end of respective elements 84. The opposite ends of elements 84 are coupled to respective portions of a linkage 96 in forward section 74. The linkage is preferably coupled to endoscope 80 near the tip of distal portion 82. Movement of pistons 90 is thus translated to linkage 96, and differential motion of pistons 90 causes rotation of linkage 96 and turning of the distal end of the endoscope at joint 76 (FIG. 1B). As appropriate, linkage 96 may be shaped like a ring. Alternatively, the linkage comprises one or more connection points of elements 84 to the forward section. Seals 99 are preferably provided to facilitate the movement of elements 84 while inhibiting leakage of fluid from cylinders 88. Those of skill in the art will appreciate that there are a variety of means for coupling elements 84 in order to produce desired motions of endoscope 80. For example, elements 84 may be rigid or flexible, and coupled to the other elements of distal portion 82 so as to be placed in compression or tension responsive to the pressure in cylinders 88.

Movement of pistons 90 is driven by fluid delivered to or withdrawn from cylinders 88 via flexible tubes 86. It is to be understood in the context of the present patent application and in the claims that the term “fluid” is meant to include a liquid and/or a gas. Preferably, each cylinder 88 is aligned parallel to the longitudinal axis of the endoscope, and fluid is delivered to or withdrawn from a port 89 near the distal end of the cylinder. Each cylinder is thus divided into two sections by piston 90: (a) a fluid transfer section 91, closer to the distal end of the endoscope, where fluid is delivered or withdrawn, and (b) a passive section 93, closer to the proximal end of the endoscope.

Advantageously, linkage 96 is preferably designed to operate responsive to tension (or compression) in one of elements 84, whereby applying positive pressure to one piston 90 tends to force that piston proximally. This minimizes the need for suction and the potential problems with collapse of flexible tubes 86. Additionally, suction as a means for generating useful motion of the endoscope is generally limited to one atmosphere, while positive pressure can exceed one atmosphere. Experiments performed using the principles of the present invention have generated positive pressures of 50 atmospheres at the distal end, using only the force easily generated by hand, applied to the simple and inexpensive apparatus preferred in accordance with these embodiments of the present invention. It is emphasized that prior art systems for hydraulic endoscopic steering generally require complicated and expensive apparatus, which utilize pumps and pressure-regulation apparatus or other powered equipment to operate.

Typically, passive section 93 of cylinder 88 comprises a venting port 101, which allows fluid to enter or leave the cylinder as piston 90 is displaced. For some applications, passive section 93 of cylinder 88 is sealed, and encloses a compressible fluid such as air, which acts like a spring when piston 90 is displaced, tending to return the piston to its equilibrium position. In a preferred embodiment of the present invention, the passive section of cylinder 88 comprises an elastic element such as a spring (not shown), which returns piston 90 in its equilibrium position.

Fluid is delivered to or withdrawn from one of cylinders 88 responsive to the operation of pressure apparatus 95, preferably comprising, to control each cylinder 88, a drive-piston 94 in a drive cylinder 92. Each drive-piston 94 is preferably coupled to the respective cylinder 88 by one of flexible tubes 86. Applying a force to drive-piston 94 pressurizes the fluid in drive cylinder 92. This pressure is transmitted through the fluid in tube 86 and in cylinder 88, and comes to act on piston 90, to cause steering of endoscope 80 as described hereinabove. The ratio of the driving force applied to drive-piston 94 to the pressure force received by piston 90 is generally proportional to the area ratio of the two piston faces. Thus, fine control of the steering of endoscope 80 can be achieved by decreasing the area of piston 94 relative to the area of piston 90. In this manner, operator-induced motions of piston 94 can be leveraged to yield fine motions of piston 90. The force required to steer the endoscope can be selected by sizing drive-piston 94 and piston 90 appropriately.

In some preferred embodiments of the present invention, a mechanical linkage, such as a joystick 102 mechanically coupled to pistons 94, is used to actuate drive-pistons 94 to make steering the endoscope more ergonomic. For applications in which more cylinders are used at the distal and/or proximal ends of endoscope 80, appropriate changes in the linkage are provided, so as to facilitate greater ease of use for the operator.

In a preferred embodiment of the present invention, endoscope 80 is propelled through the gastrointestinal tract using methods and apparatus described in PCT Patent Publication WO 02/19886, entitled “Double sleeve endoscope,” which is assigned to the assignee of the present patent application and is incorporated herein by reference. As described in that application, inflation by compressed air of a dual-sleeved, extendable tube that is attached to the distal portion of an endoscope pushes the distal portion through the gastrointestinal tract of a patient without the use of wires, which are commonly used in the prior art. The absence of wires running the length of the endoscope leads to a more flexible endoscope, which does not risk buckling of members in compression, and which reduces the risk of injuring the intestine through which the endoscope travels. Preferably, use of the inflating sleeve in combination with the methods and apparatus of the present patent application allows the operator to propel and steer the endoscope around turns in the gastrointestinal tract.

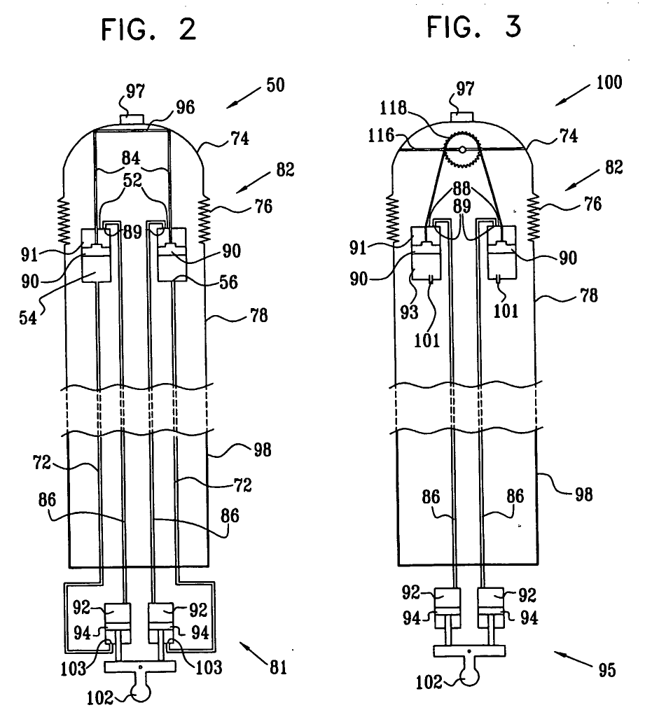

FIG. 2 is a schematic sectional drawing of a flexible endoscope 50, representing another preferred embodiment of the present invention. Preferably, endoscope 50 comprises a plurality of pistons and cylinders, generally as used in pressure apparatus 95 of FIGS. 1A and 1B, except with differences as described herein. The hydraulic steering mechanism of endoscope 50 differs from that of endoscope 80 in that fluid pressure is supplied to both faces of pistons 90 so as to provide a net force which produces motion of the pistons, while fluid pressure is only supplied to one side of pistons 90 in endoscope 80.

Each of two cylinders 52 in distal portion 82 of endoscope 50 is coupled to two fluid supply tubes coming from pressure apparatus 81: (a) a distal fluid supply tube 86 coupled to the distal end of cylinder 92, and (b) a proximal fluid supply tube 72, coupled to a port 103 on the proximal end of cylinder 92. With this closed-loop arrangement, it is possible to precisely control the net force on each piston 90, and to accurately steer the endoscope. Either a compressible fluid (e.g., air) or a substantially-incompressible fluid (e.g., water) is used to drive pistons 90.

It is to be understood that four cylinders are not required to achieve the benefits of the present invention. Closed-loop steering mechanisms similar to that shown in FIG. 2, but having fewer cylinders may be particularly useful for some applications. In a preferred embodiment, a steering mechanism is provided comprising two proximal cylinders and one distal cylinder, each of the proximal cylinders coupled by a flexible tube to apply positive pressure to opposing faces of a piston in the distal cylinder. Alternatively, a steering mechanism comprising two distal cylinders and one proximal cylinder is provided, in which tubes coupled to opposing ends of the proximal cylinder convey positive pressure to one or the other of the distal cylinders, responsive to the direction of motion of a piston in the proximal cylinder.

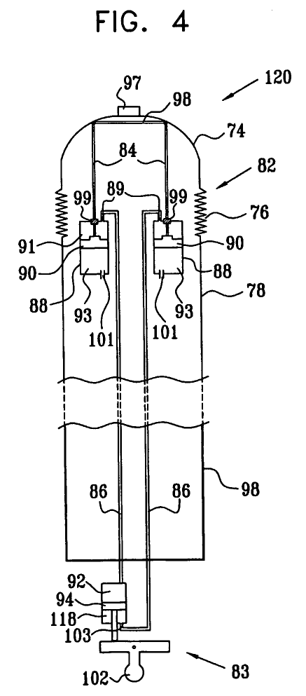

FIG. 3 is a schematic sectional drawing of a flexible endoscope 100 representing still another preferred embodiment of the present invention. Endoscope 100 functions generally similarly to endoscope 80 described hereinabove with reference to FIGS. 1A and 1B, but differs in some aspects of the steering mechanism. For endoscope 80, pistons 90 are indirectly coupled through elements 84 and linkage 96, while for endoscope 100 a substantially inextensible belt 104 directly couples opposing pairs of pistons 90. Pistons 90 are thus constricted to move equal distances in opposite directions. It is to be understood that while FIG. 4 presents only two cylinders 88, the scope of the present invention includes other embodiments comprising an even number of cylinders 88, where pairs of opposing cylinders are coupled by a belt.

Belt 104 preferably runs over a gear 118 or analogous mechanism, which is coupled to a linkage 116, such that rotation of the gear due to movement of belt 104 causes rotation of the linkage and the desired turning of the distal end of the endoscope.

FIG. 4 is a schematic sectional drawing of a flexible endoscope 120 representing yet another preferred embodiment of the present invention. Endoscope 120 functions generally similarly to endoscope 80 described hereinabove with reference to FIGS. 1A and 1B, but differs in some aspects of the pressure apparatus. Whereas pressure apparatus 95 of endoscope 80 comprises two proximal piston/cylinders, one to drive each of the two distal pistons, a pressure apparatus 83 of endoscope 120 comprises only 1 proximal piston/cylinder device. A piston 94 divides the cylinder into a distal portion 92 and a proximal portion 118, such that distal movement of piston 94 simultaneously forces fluid out of distal portion 92 and into proximal portion 118. Distal portion 92 and proximal portion 118 are each coupled to a separate cylinder 88 by separate tubes 86, so as to steer the endoscope as discussed hereinabove with reference to FIGS. 1A and 1B.

In another preferred embodiment, only one distal cylinder 88 is used to steer the endoscope, resulting in a system comprising one distal cylinder and one proximal cylinder.

It will be appreciated that the preferred embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. Endoscopic steering apparatus, comprising: an endoscope, having distal and proximal ends thereof; at least one proximal cylinder, disposed in a vicinity of the proximal end of the endoscope; at least one proximal piston, slidably coupled to the at least one proximal cylinder; a first distal cylinder, disposed at the distal end of the endoscope; a first distal piston, slidably coupled to the first distal cylinder; a second distal cylinder, disposed at the distal end of the endoscope; a second distal piston, slidably coupled to the second distal cylinder; a first tube, coupled to the first distal cylinder and to the at least one proximal cylinder; a second tube, coupled to the second distal cylinder and to the at least one proximal cylinder; and a linkage disposed at the distal end of the endoscope and coupled to the first distal piston and to the second distal piston, such that displacement of at least one of the distal pistons causes displacement of the linkage and steering of the distal end of the endoscope.

2. Apparatus according to claim 1, wherein the at least one proximal piston is adapted to be manually driven.

3. Apparatus according to claim 1, wherein the at least one proximal piston is adapted to be power driven.

4. Apparatus according to claim 1, wherein the at least one proximal cylinder comprises respective first and second proximal cylinders, wherein the at least one proximal piston comprises respective first and second proximal pistons, slidably coupled to the first and second proximal cylinders, respectively, wherein the first tube is coupled to the first distal cylinder and to the first proximal cylinder, and wherein the second tube is coupled to the second distal cylinder and to the second proximal cylinder.

5. Apparatus according to claim 1, wherein the at least one proximal cylinder comprises a single proximal cylinder, wherein the at least one proximal piston comprises a single proximal piston, slidably coupled to the single proximal cylinder, wherein the single proximal cylinder has a proximal port and a distal port, disposed at respective ends of the single proximal cylinder, and wherein the first and second tubes are coupled to the single proximal piston at the proximal and distal ports, respectively.

6. Apparatus according to claim 1, wherein the linkage comprises a flexible element, disposed in the endoscope such that tension in the element translates a displacement of one of the distal pistons into a change in angular disposition of the distal end of the endoscope.

7. Apparatus according to claim 1, wherein the linkage comprises an element, disposed in the endoscope such that compression in the element translates a displacement of one of the distal pistons into a change in angular disposition of the distal end of the endoscope.

8. Apparatus according to any one of claims 1, wherein the linkage is configured so as to translate a displacement of one of the distal pistons into a displacement of the other one of the distal pistons.

9. Apparatus according to any one of claims 1, and comprising a mechanical user-interface device, which is coupled to the at least one proximal cylinder so as to mechanically transduce a force generated by a user of the steering apparatus into a motion of the at least one proximal piston.

10. Endoscopic steering apparatus, comprising: an endoscope, having distal and proximal ends thereof, the distal end comprising a forward section and a rear section flexibly coupled to the forward section; a distal cylinder, disposed at the rear section of the distal end of the endoscope; a distal piston, slidably coupled to the distal cylinder and coupled to the forward section of the distal end of the endoscope; a proximal cylinder, disposed in a vicinity of the proximal end of the endoscope; a proximal piston, slidably coupled to the proximal cylinder; and a tube, coupled between the distal cylinder and the proximal cylinder, such that displacement of the proximal piston generates a pressure in the tube capable of displacing the distal piston and rotating the forward section with respect to the rear section.

11. Apparatus according to claim 10, wherein the proximal piston is adapted to be manually driven.

12. Apparatus according to claim 10, wherein the proximal piston is adapted to be power driven.

13. Apparatus according to claim 10, and comprising a mechanical user-interface device, which is coupled to the proximal cylinder so as to mechanically transduce a force generated by a user of the steering apparatus into a motion of the proximal piston.

14. Apparatus according to claim 10, wherein the distal cylinder has a distal port, distal to the distal piston, in communication with the tube, such that positive pressure in the tube responsive to displacement of the proximal piston induces proximal motion of the distal piston.

15. Apparatus according to claim 14, wherein the proximal cylinder has a first port, which is in communication with a first face of the proximal piston, and a second port, which is in communication with a second face of the proximal piston, wherein the tube is coupled to the proximal cylinder at the first port, so as to be in communication with the first face of the proximal piston, wherein the distal cylinder has a proximal port, proximal to the distal piston, wherein the apparatus comprises an additional tube having distal and proximal ends thereof, the additional tube being in communication at the distal end thereof with the proximal port of the distal cylinder, and being in communication at the proximal end thereof with the second port of the proximal cylinder, such that positive pressure in the additional tube responsive to displacement of the proximal piston induces distal motion of the distal piston.

16. Apparatus according to claim 14, wherein the apparatus comprises an additional proximal cylinder, disposed in a vicinity of the proximal end of the endoscope, wherein the apparatus comprises an additional manually-driven proximal piston, slidably coupled to the additional proximal cylinder, wherein the distal cylinder has a proximal port, proximal to the distal piston, wherein the apparatus comprises an additional tube, coupled between the proximal port of the distal cylinder and the additional proximal cylinder, such that positive pressure in the additional tube responsive to displacement of the additional proximal piston induces distal motion of the distal piston.
